(19) United States
(12) Patent Application Publication
(10) Pub. No.: US 2025/0205055 A1
(43) Pub. Date: Jun. 26, 2025

(54) CADAVERIC 6-AXIS SPINE SIMULATOR FOR ASSESSING SPINAL BIOMECHANICS

(71) Applicant: UNIVERSITY OF SOUTH FLORIDA, Tampa, FL (US)

(72) Inventor: Nathan Schilaty, Tampa, FL (US)

(21) Appl. No.: 18/394,762

(22) Filed: Dec. 22, 2023

(51) Int. Cl. A61F 2/28; G09B 23/30

(52) U.S. Cl. CPC A61F 2/2857; G09B 23/30

(57) ABSTRACT

A spine simulator is disclosed herein. This spine simulator may be used to test and assess the biomechanical effects of various interventions to the spine that include surgical implants, excision, or fusion. Surgical implants, excision, or fusion can change the biomechanical outcomes of the specific spinal segments as recorded by load cells, 3D motion capture, and sensors, such as displacement sensors included in the spine simulator. These biomechanical effects are best measured in the spine with cadaveric specimens with multiple instrumentations that allow for complex movement analysis. Preferably the spine simulator is capable of customized testing of spinal segments from the cervical to sacral spine. Related methods of use are also provided.

### FIELD OF THE INVENTION

The disclosed technology is generally directed to a biomechanics simulator. More particularly the technology is directed to a spinal simulator.

### BACKGROUND

The spinal column is an integral part of the human body that houses the spinal cord and allows for trunk flexibility. The spine is subject to normal age-related processes that cause degeneration, pain, and disability. In fact, lower back pain is the leading cause of disability and the highest expenditure of the musculoskeletal system for health care. Further, greater than 80% of adults will experience low back pain at some point in their lifetime. Neck pain and disability are close behind for musculoskeletal expenditure and disability.

Biomechanical simulations can be performed to study the induced changes from various interventions and injuries. The benefit of simulations is the ability to instrument specimens and discover answers to questions that would otherwise be impossible with an in vivo application. Currently, simulators are capable of studying 3-7 motion segments. There is a requisite need to study the biomechanics of the full spine as biomechanics can globally affect the spine and determine improved interventions that will decrease both the disability and pain load of the worldwide population to this musculoskeletal disease burden.

### BRIEF SUMMARY

Disclosed herein is a spine simulator including a frame, a set of struts extending from the frame, a gimbal assembly slidably connected to the frame, a base assembly slidably connected to the set of struts, where the base assembly is approximately aligned with the gimbal assembly, a first spine support on the base configured to receive a first end of a specimen spine, a second spine support on the gimbal assembly configured to receive a second end of the specimen spine, a set of pneumatic cylinders arranged to apply a force to the base, an electronic controller, and a plurality of sensors positioned in or on the specimen spine and coupled to the electronic controller, where the gimbal assembly includes at least three rotary components and at least three sliding components, wherein the base assembly comprises a load cell, where the electronic controller is coupled to the gimbal assembly, the base assembly, the set of pneumatic cylinders, the at least three rotary components, and the load cell to send instructions and receive data, and where the distance between the gimbal assembly and the base can be varied to accommodate a specimen spine having a length of from 2 vertebrae to 33 vertebrae with or without a pelvis. The specimen spine may be a portion of a spine, for instance any number of vertebrae from 2 to 33, e.g., 2, 3, 4, 5, 6, . . . 29, 30, 32, 33, etc.

In an aspect, the second spine support can be independently rotated by any one of the three rotary components. In another aspect, the second spine support can be independently translated by any one of the three sliding components. In yet another aspect, the plurality of sensors includes at least one of an extensometer, a strain sensor, a linear displacement sensor, and a pressure catheter positioned within at least one disc. In a further aspect, each of the at least three rotary components is driven by a dedicated stepper motor. In another aspect, at least one of the rotary components includes a harmonic strain wave gearing assembly to produce torque from the stepper motor. In an additional aspect, the spine simulator includes a 3D motion system, wherein the 3D motion system comprises a set of cameras positioned to take and send images and/or video of the frame with the specimen spine from at least two positions.

In an aspect, the base assembly further includes a pelvis fixture that includes a bracket with two mock femurs and a hook extending from the bracket, wherein the pelvis fixture receives a pelvis, and wherein the pelvis receives the two mock femurs at the acetabulum and the hook secures the pelvis at the pubic symphysis. The hook of the pelvis fixture is moveably coupled to the bracket. In an aspect, the spine simulator further includes a muscle actuator coupled to the specimen spine. In another aspect, the spine simulator includes a pulley system coupling the muscle actuator to a muscle on the specimen spine.

Further disclosed herein is method of simulating spine motion including the following steps: (a) receiving a specimen spine having a first end and a second end and a plurality of sensors, (b) fixing the first end in a first spine support and fixing the second end in a second spine support to arrange the specimen spine in a starting position, and (c) subjecting the first spine support and/or the second spine support to at least one force selected from a translational force or a force effected by at least one of a set of pneumatic cylinders or rotary components and returning the specimen spine to the starting position, where the at least one force is applied between 2 and 2,000,000 repetitions and the specimen spine is returned to the starting position between each repetition, and where a controller collects data from the plurality of sensors for each repetition to generate a first data set. The forces may be applied in any or all of an X, Y, or Z axis as shown in FIG. 2, as well as rotational forces. In an aspect, one end of the spine may be held in a fixed position while the opposite end is moved, or both ends may be moved, for instance, moved together (compressing the spine), moved apart (extending the spine), rotated, tilted, moved laterally relative to one another, and combinations thereof. In an aspect, the method further includes the steps of: (d) performing an intervention on the specimen spine, and (e) resubjecting the specimen spine to the at least one force and repeating the application of the at least one force between 2 and 2,000,000 repetitions, and where the controller collects data from the plurality of sensors for each repetition to generate a second data set.

In one aspect, the application of the at least one force effects a motion of the specimen spine. In another aspect, the motion is a coupled motion comprising at least two of flexion, extension, lateral flexion, or rotation. In yet another aspect, the force is applied to a muscle of the specimen spine via a pulley system activating a muscle actuator. In a further aspect, the force applied can be dynamically altered according to data captured from in vivo test subjects to mimic physiologic conditions.

In one aspect, the intervention is at least one of introducing a surgical implant, excision, and fusion. In another aspect, the motion comprises application of negative pressure by the pneumatic cylinders. In a further aspect, the specimen spine has a length between 2 vertebrae and 33 vertebrae with a pelvis.

Further disclosed herein is a spine simulator including a gimbal assembly, a base approximately aligned with the gimbal assembly, a first spine support on the base configured to receive a first end of a specimen spine, a second spine support on the gimbal assembly configured to receive a second end of the specimen spine, a set of pneumatic cylinders arranged to apply a force to the base, an electronic controller, and a plurality of sensors positioned in or on the specimen spine and coupled to the electronic controller, where the gimbal assembly comprises at least three rotary components to rotate the second spine support and at least three sliding components to translate the second spine support, where the base comprises a load cell, wherein the electronic controller is coupled to the gimbal assembly, the base, the pneumatic cylinders, the rotary components, and the load cell to send instructions and receive data, and where the distance between the gimbal assembly and the base can be varied to accommodate a specimen spine having a length between 2 vertebrae and 33 vertebrae including with or without a pelvis.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows the structural features of the spine simulator with an illustrative full spine and pelvis.

FIG. 2 shows a top perspective view of the spine simulator including the gimbal assembly and the base assembly, according to an aspect of the disclosure herein.

FIG. 3 shows a bottom perspective view of the spine simulator including the gimbal assembly and the base assembly, according to an aspect of the disclosure herein.

FIG. 4A shows a right-side view, 4B a left-side view, and 4C a front view of the spine simulator including the gimbal assembly and the base assembly, according to an aspect of the disclosure herein.

FIG. 5A and 5B show detailed, perspective views of the gimbal assembly, according to an aspect of the disclosure herein.

FIG. 6 shows an example of a rotary component suitable for use with the gimbal assembly, according to an aspect of the disclosure herein.

FIG. 7 shows a single rotary component, according to an aspect of the disclosure herein.

FIG. 8 shows a detailed, perspective view of the base assembly of FIG. 2, according to an aspect of the disclosure herein.

FIG. 9 shows a pelvis fixture designed to be used with the spine simulator of FIG. 2, according to an aspect of the disclosure herein.

FIG. 10 shows the pelvis fixture of FIG. 9 with a pelvis attached, according to an aspect of the disclosure herein.

FIG. 11 shows a schematic of an arrangement of a spine simulator with a 3D motion system including tripod mounted cameras situated near the spine simulator.

FIG. 12 shows an example of a muscle actuator.

### DETAILED DESCRIPTION

Documented changes of biomechanical outcomes can provide for change of clinical practice, either through surgical modification, new device development, or by management of patient interventions prior to surgical intervention. To address this need, a spine simulator 100 is disclosed herein. This spine simulator 100 is designed to help assess the biomechanical effects of various interventions to the spine that include surgical implants, excision, or fusion. Surgical implants, excision, or fusion can change the biomechanical outcomes of the specific spinal segments as recorded by load cells, 3D motion capture, and displacement sensors included in the spine simulator 100. These biomechanical effects are best measured in the spine with cadaveric specimens with multiple instrumentations that allow for complex movement analysis. This work will inform biomechanical changes that may improve surgical practice, spinal pain mechanics, outcomes of interventions, and patient satisfaction. The spine simulator 100 is capable of customized testing of spinal segments from the cervical to sacral spine.

### Spine Simulator Structure

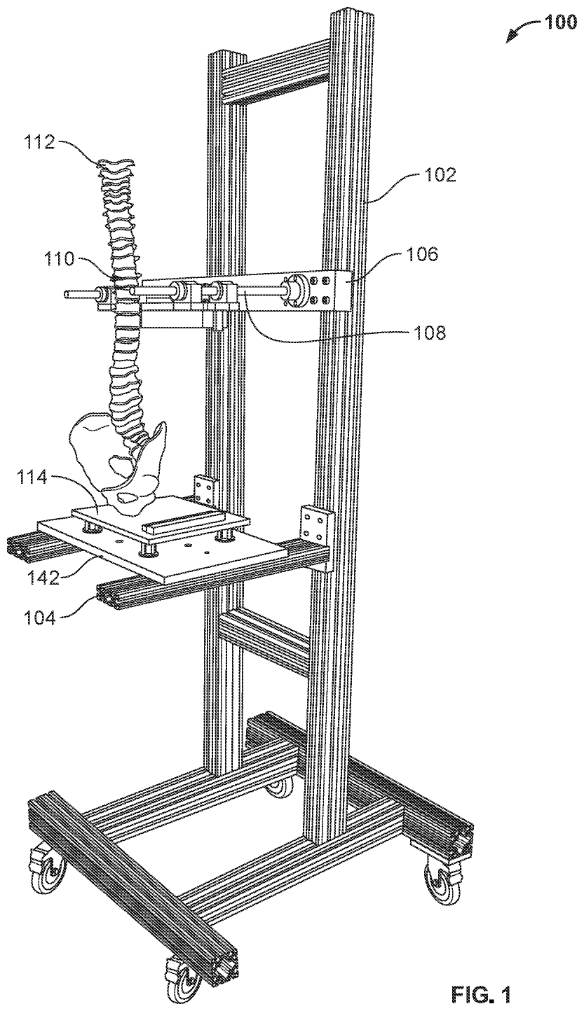

FIG. 1 shows structural features of one example of the spine simulator 100. Structural features of the spine simulator 100 can include a frame 102 with a set of struts 104 extending from the frame 102. The spine simulator 100 can further include a plate 106 with connected support arms 108. The frame 102 can be a free-standing frame. Additionally, and alternatively, the spine simulator 100 and/or selected components thereof can be attached to a wall, ceiling, post, floor, or other stabilizing structure. In the non-limiting example shown in FIG. 1, the spine simulator 100 is arranged in a vertical configuration.

FIG. 1 further demonstrates one possible positioning of a specimen spine 110 relative to the structure of the frame 102. The specimen spine 110 includes a first end 112 and a second end 114. In this example, the first end 112 includes an attached pelvis, however, the specimen spine 110 can include any desired spinal segments ranging from 2 vertebrae to a complete spine with up to 33 vertebrae and an attached pelvis. It is contemplated that structural features can be positioned such that the specimen spine 110 can be held at any angle relative to the ground surface.

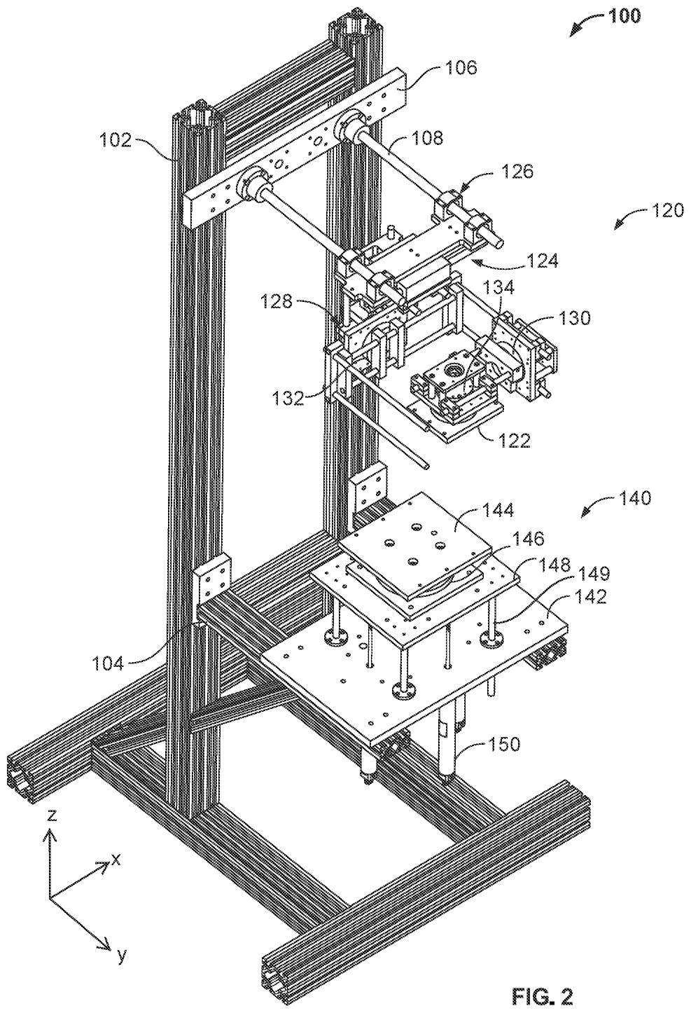

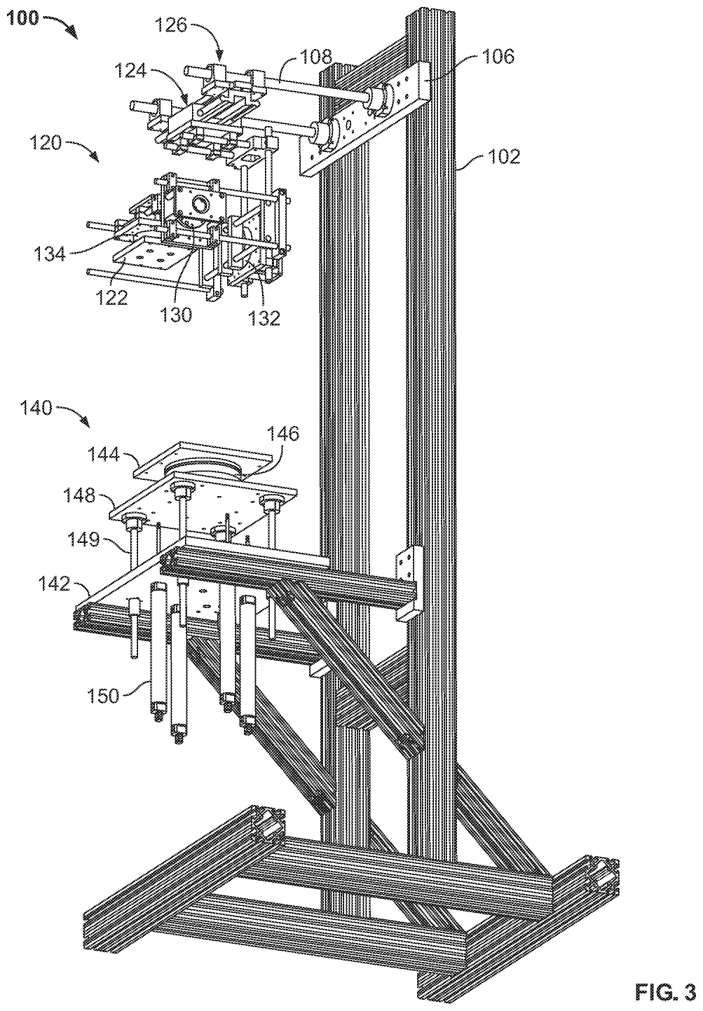

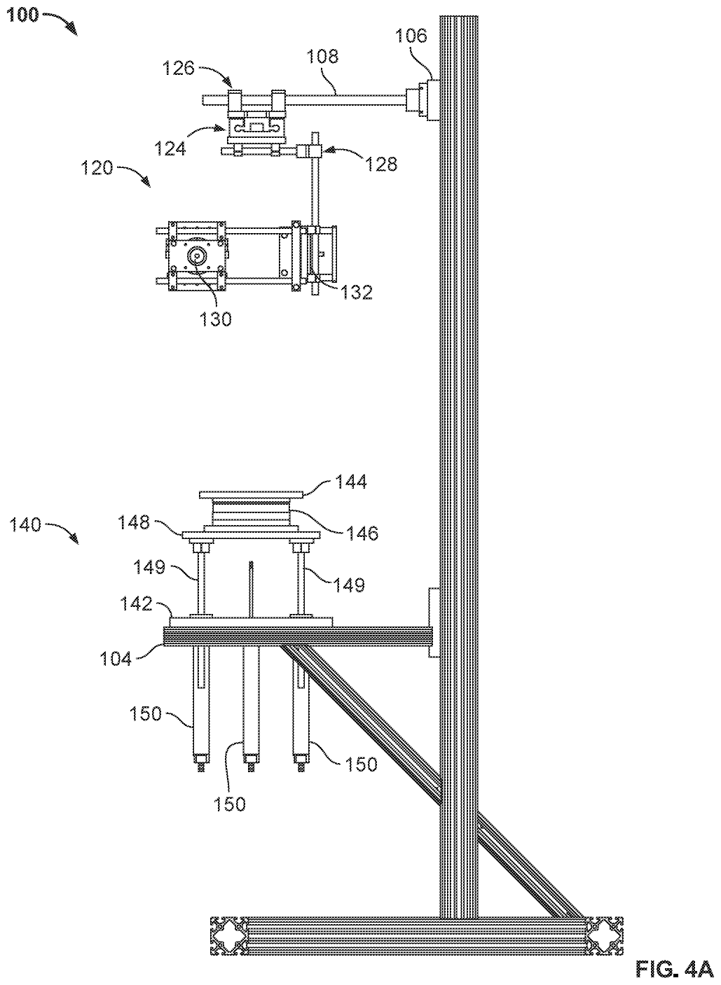

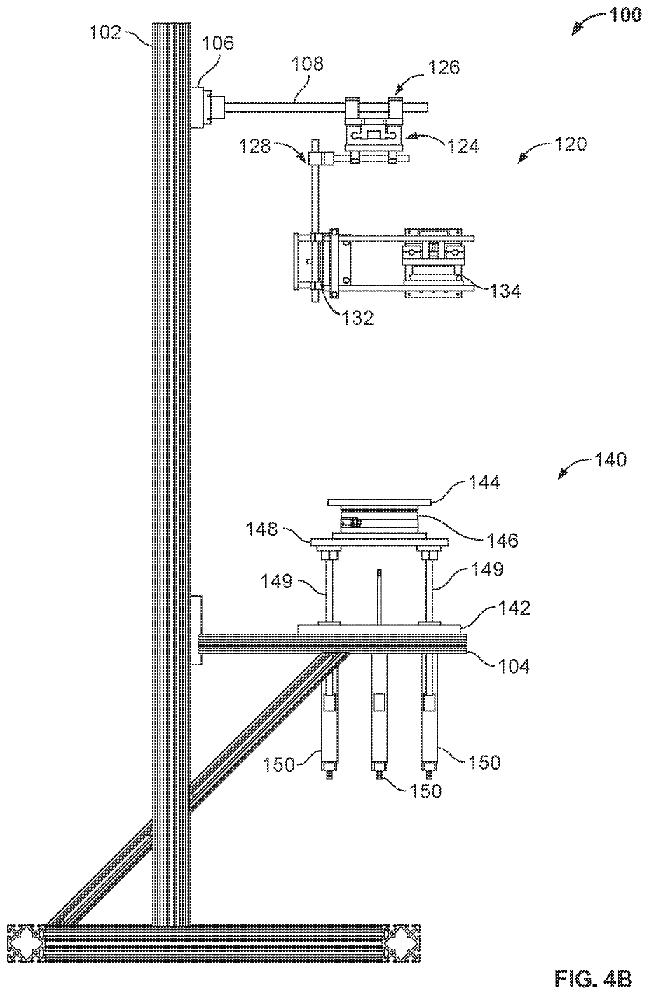

FIGS. 2-4A-4C show perspective views and side and front views of the spine simulator 100. The spine simulator 100 can include a gimbal assembly 120 that is spaced from a base assembly 140. The gimbal assembly 120 may be approximately aligned with the base assembly 140. The spacing can be adjusted as desired to accommodate different specimen spines 110. The base assembly 140 and the gimbal assembly 120 are configured to securely hold the first end 112 and second end 114, respectively.

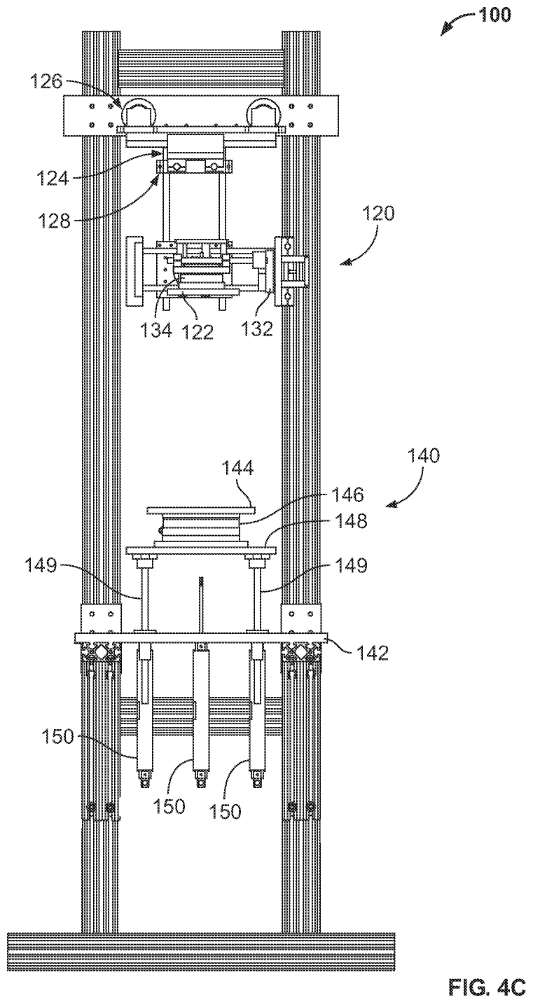

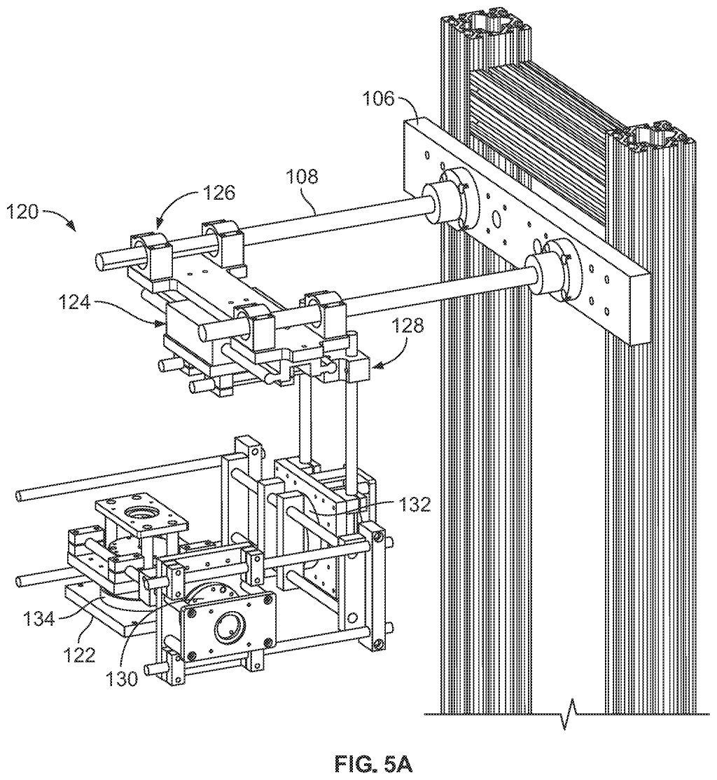

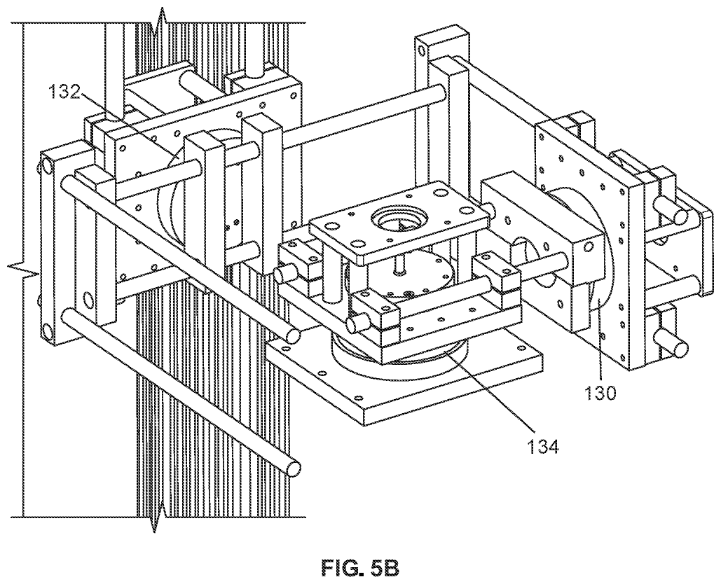

Turning to FIGS. 5A and 5B, the gimbal assembly 120 is shown in greater detail. The gimbal assembly 120 can be slidably attached to the frame 102 at the plate 106. The support arms 108 may moveably bear the gimbal assembly 120, allowing the gimbal assembly 120 to move relative to the frame 102, for example along the y-axis as shown in FIG. 2. In one example, the frame 102 can include tracks or grooves. The plate 106 can slide along the tracks and secured at a desired position. The plate 106 can be moved manually or driven by a motor controlled by an electronic controller (not shown). The plate 106 can secured at a desired position with a fastener such as a bolt or screw. By adjusting the position of the plate 106, the spacing between the gimbal assembly 120 and the base assembly 140 can be adjusted to the desired distance to accommodate the specimen spine 110.

The gimbal assembly 120 may include a second spine support, or a second end mount 122 to receive and secure the second end 114 of the specimen spine 110. The second end 114 can be immobilized or “potted” in a second end holder 118 (not shown). The second end holder 118 can be any type of suitable fixture for fastening the second end 114 to the second end mount 122. In one example, the second end holder 118 is a cylindrical pot, where the second end 114 is immobilized by use of a hardening polymer or other suitable fastening means. The second end holder 118 can be fastened on the second end mount 122.

The gimbal assembly 120 couples the second end 114 to sliding mechanisms and rotary mechanisms. The gimbal assembly 120 can include one or more or at least three sliding mechanisms, for example a first sliding mechanism 124, a second sliding mechanism 126, and a third sliding mechanism 128. The first sliding mechanism 124 can move the second end mount 122 in the x-axis. The second sliding mechanism 126 can move the second end mount 122 in the y-axis. The third sliding mechanism 128 can be activated to move the second end mount 122 along the z-axis. The at least three sliding mechanisms can be moved manually or can be controlled independently or together by an electronic controller 138 (not shown). In some examples, the sliding mechanisms 124, 126, 128 can be low friction precision slides. Movements of the sliding mechanisms 124, 126, 128 are transmitted to the second end 114 of the specimen spine 110 as the second end holder 118 is secured to the second end mount 122. Motors or other actuators may be provided to move the sliding mechanisms.

Further included in the gimbal assembly 120 are one or more or at least three rotary components that can effect rotations of the specimen spine 110 or impart rotational forces thereon. For example, the spine simulator 100 can include a first rotary component 130 for rotation about the x-axis, a second rotary component 132 for rotation about the y-axis, and a third rotary component 134 for rotation about the z-axis. The rotary components can each include an independent motor (not shown) such as a stepper motor or other actuator or movement mechanism for driving the rotation. The at least three rotary components 130, 132, 134 can be driven independently or together. The at least three rotary components 130, 132, 134 can be driven in combination with the sliding mechanisms 124, 126, 128 to effect coupled motions. The rotary components can be operated manually or can be driven by the electronic controller. Movements of the rotary components 130, 132, 134 are preferably transmitted to the second end 114 of the specimen spine 110 as the second end holder 118 is secured to the second end mount 122.

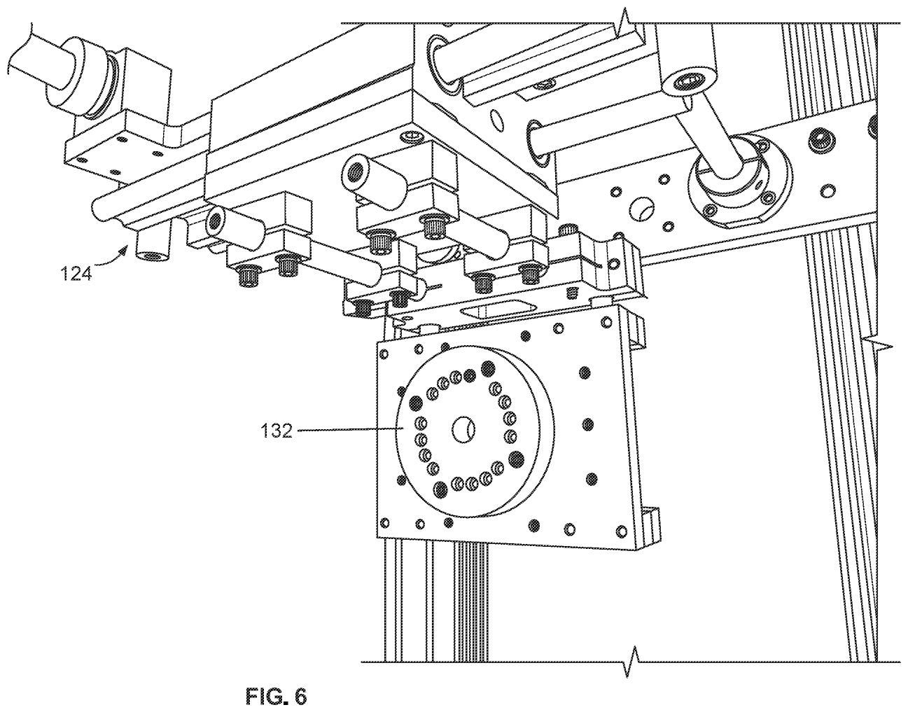

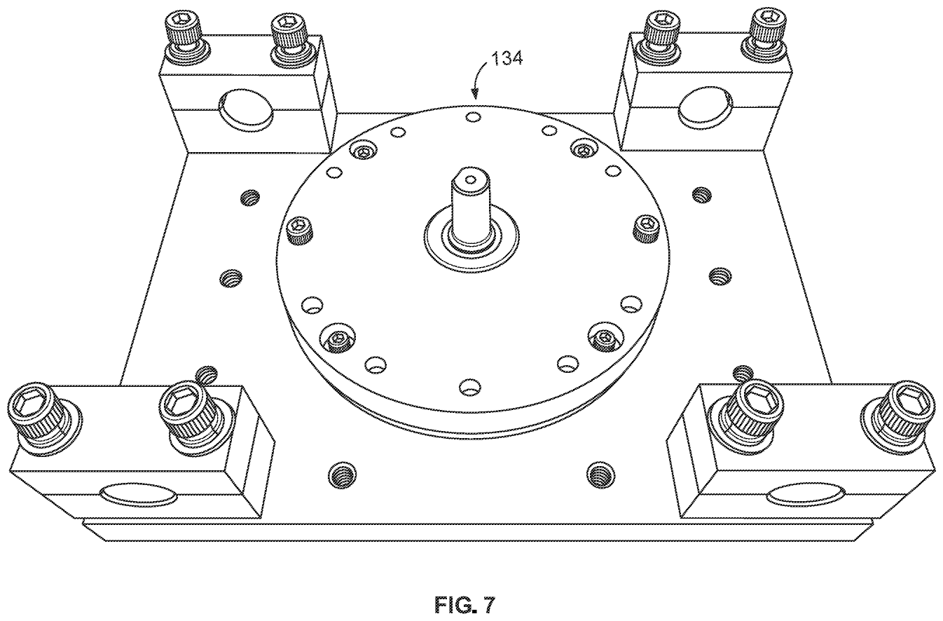

The gimbal assembly 120 according to an aspect is shown in FIG. 6 with a rotary component. Additionally, an example of a rotary component is shown in FIG. 7. The rotary components can include precision reduction gear units and wash down closed loop stepper motors to allow precise control of the rotation. In a particular example, the rotary components can operate at a maximum of 25 Nm of torque. Further, the rotary components can include a harmonic strain wave gearing assembly to produce torque from the stepper motor.

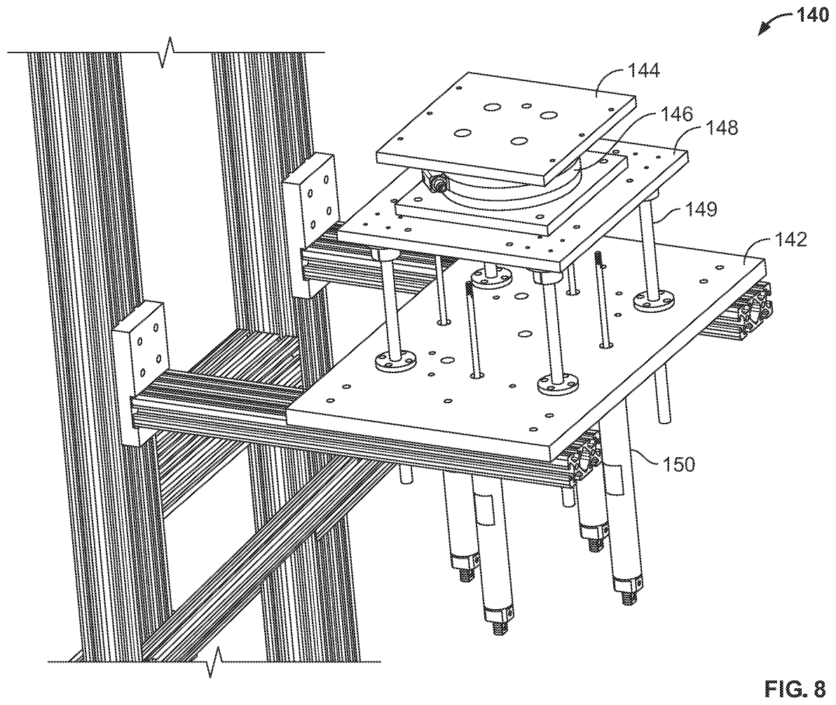

Details of the base assembly 140 are shown in the expanded view in FIG. 8. The base assembly 140 can be attached to the frame 102 by the platform 142. In the example shown, the base assembly 140 is attached to the struts 104. In one example, the struts 104 can include tracks or grooves that allow the base assembly 140 to slide along each of the struts 104. In another example, the struts 104 can include a set of spaced holes that align with a set of holes in the base assembly, such that the base can be secured at a desired position using suitable fasteners.

Included in the base assembly 140 are a first spine support, or a first end mount 144, a load cell 146, and a load cell plate 148. The first end mount 144 receives and secures the first end 112 of the specimen spine 110. Between the first end mount 144 and the load cell plate 148 is the load cell. The load cell 146 can be for example, a force torque sensor, or a device that measures the outputting forces and torques from all three coordinates (x, y, and z).

The load cell plate 148 may be moveable along the z-axis. The base assembly 140 can include a set of bars 149 for adjusting the height of the load cell plate 148 with respect to the platform 142. A set of pneumatic cylinders 150 that extend through the platform 142 to contact the load cell plate 148 can be included in the base assembly 140. The pneumatic cylinders 150 can apply a pressure to the load cell plate 148 and are controlled by the electronic controller. In some examples, the pneumatic cylinders 150 can apply a positive pressure or a negative pressure to the load cell plate 148. Suitable alternative movement mechanisms may be provided in addition to or instead of pneumatic cylinders.

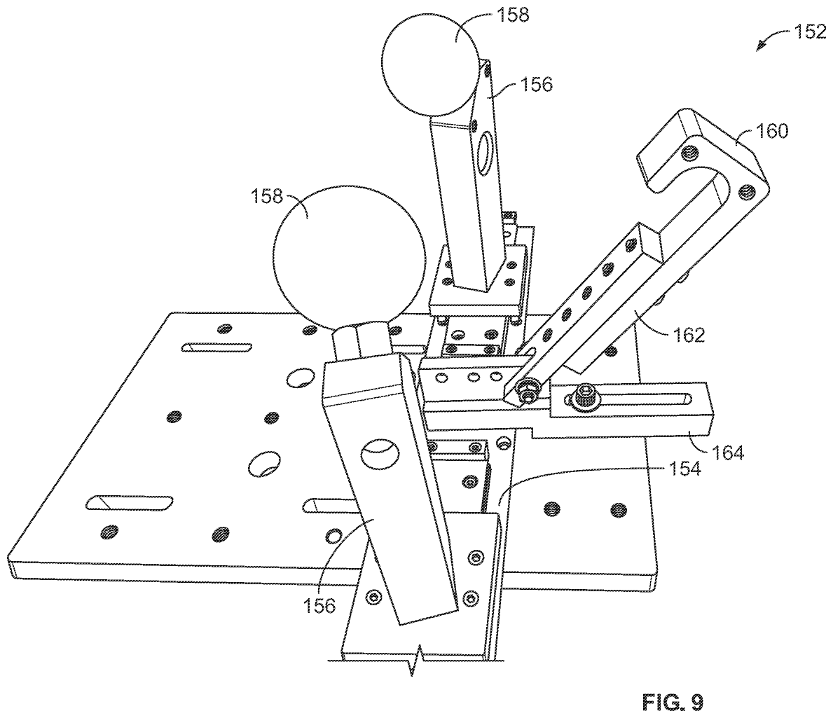

The base assembly 140 may be fastened to a pelvis fixture 152 to receive a specimen spine 110 having an attached pelvis. The pelvis fixture 152 according to an aspect disclosed herein is shown in FIG. 9. The pelvis fixture 152 includes a bracket 154. Mounted on the bracket 154 are two spaced apart mock femurs 156. The mock femurs have ball ends 158. A hook 160 extends from and is rotatably mounted to the bracket 154 such that the angle of the hook 160 with respect to the bracket 154 can be adjusted. The hook 160 further has an adjustable stem 162 that can be extended or shortened and secured at a desired length.

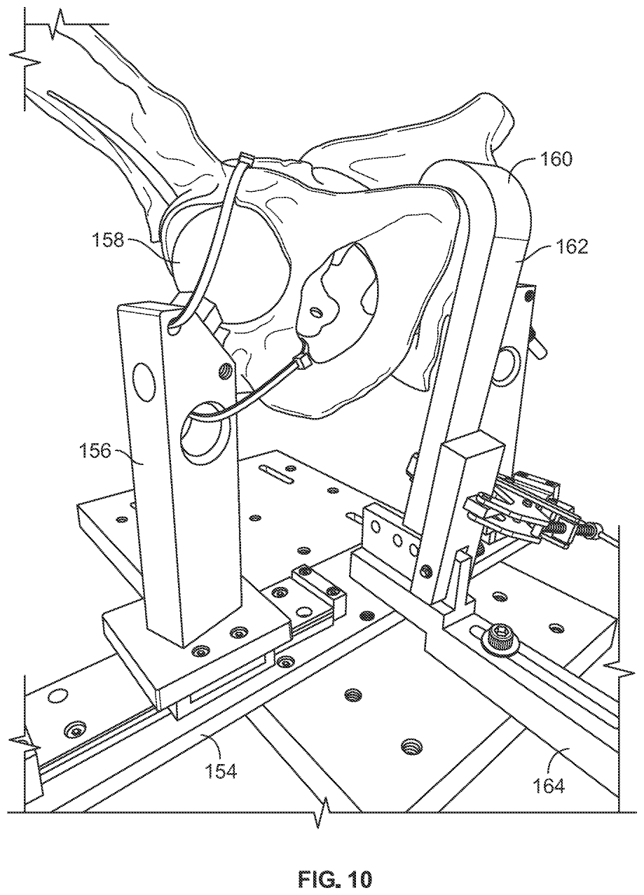

The hook 160 is positioned between the two mock femurs 156. To hold a pelvis in the pelvis fixture 152, the ball ends 158 are received by the acetabulum or cotyloid cavities of the pelvis. Additional fasteners may be used, for example zip ties as shown, to secure the pelvis to the mock femurs 156. The ball ends 158 are moveable with respect to the bracket in order to accommodate any size and shape of pelvis. The hook 160 can engage the pelvis at the pubic symphysis as shown in FIG. 10. The sacroiliac joint, between the pelvis and spine, is notoriously difficult to study. By accommodating a full spine and pelvis, the spine simulator 100 can include the sacroiliac joint in the simulations.

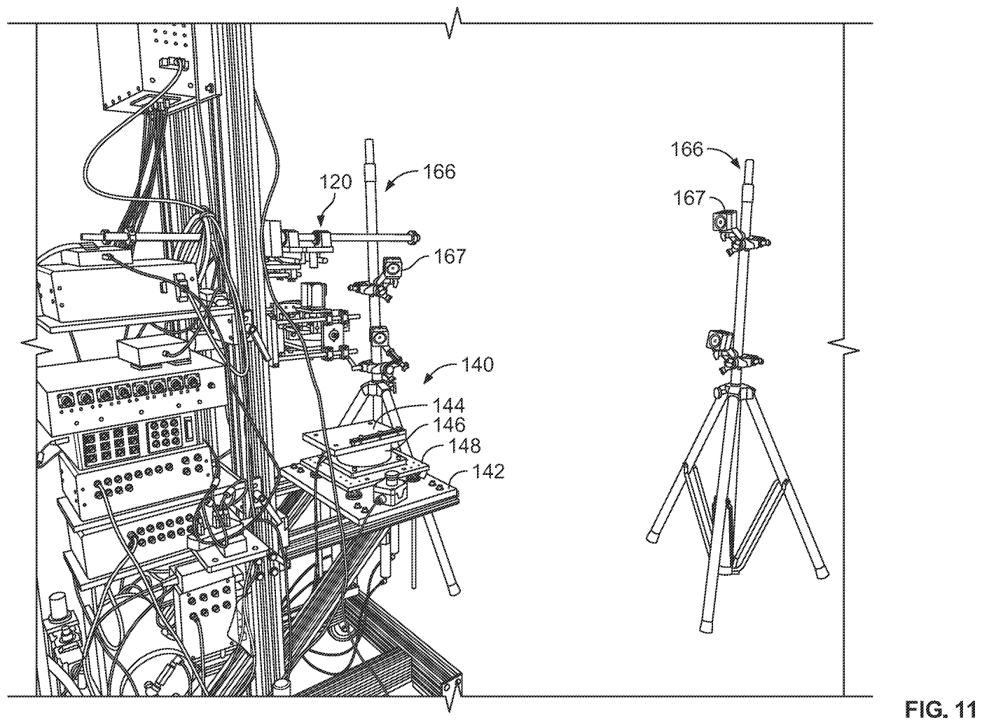

The spine simulator 100 can further include a 3D motion system 166. The 3D motion system 166 can include a set of cameras 167 positioned to take and send images and/or video and/or related data of the spine simulator 100 with the specimen spine 110 from at least two positions. One example of positioning of the 3D motion system is shown in FIG. 11. The 3D motion system may include a computer optionally with memory and software configured to determine the position and orientation of the specimen spine 110 or portions of the specimen spine 110 based on images and/or video collected by the set of cameras optionally along with data related to the images and/or video. The images and/or video and/or related data may be used to measure and characterize the response of the specimen spine 110 to forces applied thereto. The characterization may factor in the forces applied to the specimen spine 110, for instance in real time.

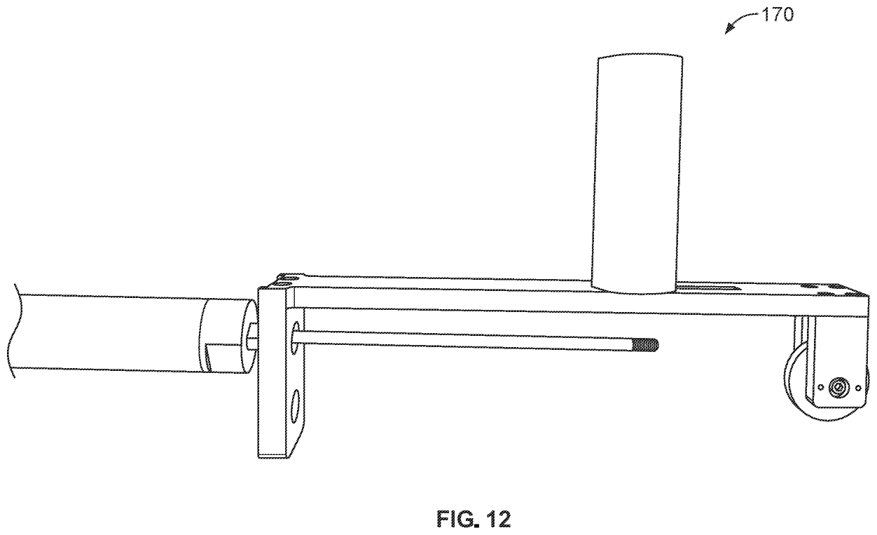

The spine simulator 100 can further include at least one muscle actuator 170. An example of a muscle actuator 170 is shown in FIG. 12. Each muscle actuator 170 may be positioned on the load cell plate 148 so as not to interfere with load measurement of spine mechanics during a simulation. In one embodiment the muscle actuator 170 utilizes carbon fiber rope on a pulley system to transfer linear load to the line of action of the respective muscle and is tethered to the tendons with a cable pulling grips and cable clamp (not shown). The muscle actuators can be affixed to load cell plate with machine screws. A cable clamp, for instance attached with a U-bolt, may be affixed to the (preferably denuded) tendons of the desired muscles. This cable clamp can then be attached to the terminal end of the pneumatic actuator to apply specific loading characteristics to the muscle tissues. The muscle actuator may be controlled by an electro-pneumatic regulator and a programmable pneumatic switch.

### Specimen Spine

The specimen spine 110 can be obtained from a cadaver, including human or animal subjects. Alternatively, the specimen spine 110 can be a porcine spine. After thawing and dissection to isolate the desired spinal segments including musculature and ligature, the specimen spine 110 can be fitted with a plurality of sensors, comprising at least one of an extensometer, a strain sensor, a linear displacement sensor, and a pressure catheter positioned within at least one disc. The specimen spine 110 can be further modified for mounting on the spine simulator 100. The first and second ends 112, 114 of the specimen spine 110 can be “potted” or immobilized in a first end holder 116 and second end holder 118, allowing the specimen spine 110 to be mounted to the spine simulator 100. If the specimen spine 110 includes a sacrum with no pelvis the sacrum can be the structure potted. However, if a full pelvis is available and the pelvis fixture 152 is used, then the sacrum can be measured by 3D motion capture with the movement of the specimen spine 110 as described below. Data collected can include: intradiscal pressure, ligamentous strain, 6 degree of freedom forces/moments of spinal segments, displacement of bony structures, and 3D motion.

Biological specimens can be used to simulate conditions that occur in vivo with surgical or other interventions. The post-mortem specimens allow measurement and analysis of data that is otherwise impossible to acquire from live human subjects. This data is useful for updating current procedures to ones that are more effective or useful for improved patient outcomes. Data acquired may allow for development of finite element models that can simulate further conditions that are not possible with cadaveric biomechanical simulations.

### Operation

In operation, a specimen spine 110 with a first end holder 116 and second end holder 118 is mounted to the spine simulator 100. The sliding mechanisms 124, 126, 128 and the rotary components 130, 132, 134 provide six degrees of freedom for moving the specimen spine 110. The specimen spine 110 can be initially evaluated without any intervention to understand baseline conditions of the spine. Once baseline parameters are understood, the selected intervention can be initiated. Non-limiting examples of interventions include excision, fusion, and introduction of an implant.

To conduct a simulation, the first spine support and/or the second spine support are subjected to at least one force selected from a translational force effected by a sliding mechanism, a force effected by at least one of the set of pneumatic cylinders, and a torque effected by at least one rotary component. The applied force effects a motion of the specimen spine 110, where the motion can be at least one of flexion, extension, lateral flexion, or rotation. For example, flexion and extension (a forward-backward motion) can be simulated by combining a sliding motion of the second end holder along the support arms 108 (y-axis) with rotation by the third rotary component 128. In another example, lateral flexion (a sideways motion) can be simulated by sliding the second end holder along the x-axis by activating the second sliding mechanism 126 and the first rotary component 130. Rotation about the z-axis can be simulated by activating the third rotary component 134. Accordingly, the simulation motion can be a coupled motion including at least two of flexion, extension, lateral flexion, or rotation. The force applied can be dynamically altered according to data captured from in vivo test subjects to mimic physiologic conditions. After the force is applied and motion has ceased, the specimen spine 110 is returned to the starting position by repositioning the holders 116, 118. In some examples, the application of force followed by return to the starting position can be repeated between 1 and 1,000 times, between 1 and 10,000 times, between 1 and 100,000 times, between 1 and 1,000,000 times, or between 1 and 10,000,000 times. The repetitions can be performed over a length of time that is less than a minute, less than one hour, between 1 hour and 24 hours, between 1 day and 1 week, between 1 week and 2 weeks, between 1 week and 4 weeks, or more than one month. The repetitions can be performed in bursts when multiple repetitions are performed, interspersed with rest times when no repetitions are performed. Data collected from the sensors during the simulation can include range of motion, kinetics, kinematics, 3D motion, pressure, displacement, and strain. The data can be saved in formats including CSV, TXT, or TDMS formatted files that contain raw data of kinetics, kinematics, displacement, pressure, 3D motion, or voltage.

In one example, a simulation can utilize a cohort of specimen spines 110 specific to the region of the desired spinal segments. Once the specimen spines 110 can be appropriately dissected and prepped for placement into the spine simulator 100, and the specimen spine 110 can be instrumented and affixed to the spine simulator 100. Baseline measurements can be performed by gathering data from the sensors as the specimen spine 110 undergoes multiple repetitions of the common spinal motions in the spine simulator 100. Thereafter, an intervention will be applied (e.g., surgery implant, excision, or fusion). After the intervention, the same measurements will be performed as at baseline to determine differences that have occurred in the biomechanics due to the intervention.

Biomechanical simulation of the spinal segments allows for testing implants for durability, identify weaknesses in current practice, and also helps improve the biomechanics of future interventions for improvement in vivo patient outcomes. The spine simulator 100 allows the complex motion of the spinal segments and their pressure distributions to the intervertebral discs to be studied, including artificial intervertebral discs, various spinal reconstruction techniques, effect of disc herniation and disc damage, and potential methods for prevention of spinal pain especially with development of finite element models.

### Miscellaneous

Unless otherwise specified or indicated by context, the terms “a”, “an”, and “the” mean “one or more.” For example, “a component” should be interpreted to mean “one or more components.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1—Biological Specimen Details

Biological specimens can be acquired through an anatomic gift registry or through an authorized FDA vendor of animal specimens (e.g., porcine spines).

The biological specimen will be stored in a −20° C. freezer until 24 hours prior to dissection. After dissection and prior to utilization on the spine simulator 100, the specimen will be stored in a dedicated biohazard refrigerator for up to ˜12 hours. If the specimen will be used again after an extended period of time (>12 hours), the specimen will again be stored in the −20° C. freezer.

Biological specimens may be stored for 2-3 months prior to testing. Once the specimen has been successfully simulated, the specimen will be discarded according to the regulations of the anatomic gift registry (typically incineration).

What is claimed is:

1. A spine simulator comprising:
a frame;
a set of struts extending from the frame;
a gimbal assembly slidably connected to the frame;
a base assembly slidably connected to the set of struts, wherein the base assembly is approximately aligned with the gimbal assembly;
a first spine support on the base configured to receive a first end of a specimen spine;
a second spine support on the gimbal assembly configured to receive a second end of the specimen spine;
a set of pneumatic cylinders arranged to apply a force to the base;
an electronic controller; and
a plurality of sensors positioned in or on the specimen spine and coupled to the electronic controller,
wherein the gimbal assembly comprises at least three rotary components and at least three sliding components,
wherein the base assembly comprises a load cell,
wherein the electronic controller is coupled to the gimbal assembly, the base assembly, the set of pneumatic cylinders, the at least three rotary components, and the load cell to send instructions and receive data, and
wherein the distance between the gimbal assembly and the base assembly can be varied to accommodate a specimen spine having a length between 2 vertebrae and 33 vertebrae with a pelvis.

2. The spine simulator of claim 1 wherein the second spine support can be independently rotated by any one of the three rotary components.

3. The spine simulator of claim 1 wherein the second spine support can be independently translated by any one of the three sliding components.

4. The spine simulator of claim 1, wherein the plurality of sensors comprises at least one of an extensometer, a strain sensor, a linear displacement sensor, and a pressure catheter positioned within at least one disc.

5. The spine simulator of claim 1, wherein each of the at least three rotary components is driven by a dedicated stepper motor.

6. The spine simulator of claim 5, wherein at least one rotary component comprises a harmonic strain wave gearing assembly to produce torque from the stepper motor.

7. The spine simulator of claim 1 further comprising a 3D motion system, wherein the 3D motion system comprises a set of cameras positioned to take and send images and/or video of the frame with the specimen spine from at least two positions.

8. The spine simulator of claim 1 wherein the base assembly further comprises a pelvis fixture that comprises a bracket, two mock femurs and a hook extending from the bracket, wherein the pelvis fixture receives a pelvis, and wherein the pelvis receives the two mock femurs at the acetabulum and the hook secures the pelvis at the pubic symphysis.

9. The spine simulator of claim 8, wherein the hook of the pelvis fixture is moveably coupled to the bracket.

10. The spine simulator of claim 1, further comprising a muscle actuator coupled to the specimen spine.

11. The spine simulator of claim 10, further comprising a pulley system coupling the muscle actuator to a muscle on the specimen spine.

12. A method of simulating spine motion comprising the following steps:
(a) receiving a specimen spine having a first end and a second end and a plurality of sensors;
(b) fixing the first end in a first spine support and fixing the second end in a second spine support to arrange the specimen spine in a starting position; and
(c) subjecting the first spine support and/or the second spine support to at least one force selected from a translational force or a force effected by at least one of a set of pneumatic cylinders or rotary components and returning the specimen spine to the starting position,
wherein the at least one force is applied between 2 and 2,000,000 repetitions and the specimen spine is returned to the starting position between each repetition,
wherein a controller collects data from the plurality of sensors for each repetition to generate a first data set.

13. The method of claim 12, further comprising the steps of:
(d) performing an intervention on the specimen spine; and
(e) resubjecting the specimen spine to the at least one force and repeating the application of the at least one force between 2 and 2,000,000 repetitions,
wherein the controller collects data from the plurality of sensors for each repetition to generate a second data set.

14. The method of claim 12, wherein the application of the at least one force effects a motion of the specimen spine.

15. The method of claim 14 wherein the motion is a coupled motion comprising at least two of flexion, extension, lateral flexion, or rotation.

16. The method of claim 12, wherein the force is applied to a muscle of the specimen spine via a pulley system activating a muscle actuator.

17. The method of claim 12, wherein the force applied can be dynamically altered according to data captured from in vivo test subjects to mimic physiologic conditions.

18. The method of claim 13, wherein the intervention is at least one of introducing a surgical implant, excision, and fusion.

19. The method of claim 13, wherein the motion comprises application of negative pressure by the pneumatic cylinders.

20. The method of claim 13 wherein the specimen spine has a length between 2 vertebrae and 33 vertebrae with a pelvis.

21. A spine simulator comprising:
a gimbal assembly;
a base assembly approximately aligned with the gimbal assembly;
a first spine support on the base configured to receive a first end of a specimen spine;
a second spine support on the gimbal assembly configured to receive a second end of the specimen spine;
a set of pneumatic cylinders arranged to apply a force to the base;
an electronic controller; and
a plurality of sensors positioned in or on the specimen spine and coupled to the electronic controller,
wherein the gimbal assembly comprises at least three rotary components to rotate the second spine support and at least three sliding components to translate the second spine support,
wherein the base assembly comprises a load cell,
wherein the electronic controller is coupled to the gimbal assembly, the base assembly, the pneumatic cylinders, the rotary components, and the load cell to send instructions and receive data, and
wherein the distance between the gimbal assembly and the base assembly can be varied to accommodate a specimen spine having a length between 2 vertebrae and 33 vertebrae with a pelvis.
